(19) United States
(12) Patent Application Publication
(10) Pub. No.: US 2025/0128030 A1
(43) Pub. Date: Apr. 24, 2025

(54) SYSTEMS AND METHODS FOR CREATING AN ACCESS PATH BETWEEN BLOOD VESSELS

(71) Applicant: inQB8 Medical Technologies, LLC, Winchester, MA (US)

(72) Inventors: Arshad Quadri, Hartford, CT (US); Jeremy Brent Ratz, Winchester, MA (US); Garrett Dallas Johnson, Costa Mesa, CA (US); Alec Rebh, Boylston, MA (US); Matthew Steven Lapointe, Framingham, MA (US); Brianna Parbattie Raghunath, Worcester, MA (US); Casey Nicole Martin, Worcester, MA (US); Audrey Bell, Wellesley, MA (US)

(21) Appl. No.: 18/918,681

(22) Filed: Oct. 17, 2024

Related U.S. Application Data
(60) Provisional application No. 63/591,376, filed on Oct. 18, 2023.

(51) Int. Cl. A61M 25/10; A61M 25/09

(52) U.S. Cl. CPC A61M 25/1025; A61M 25/0905; A61M 2025/1043

(57) ABSTRACT

Disclosed are a catheter-based “kissing balloon” systems and methods of connecting blood vessels or creating an access path between blood vessels. The system includes a first delivery or passing catheter in a first vessel and a second receiving catheter in a second vessel that cooperate to create a path between the vessels. A passing catheter balloon is arranged to mate with or “kiss” a receiving catheter balloon to align the vessels before a piercing element is extended to puncture both vessels and optionally puncture a balloon. A guidewire is delivered through a lumen of the piercing element, and a distal end of the guidewire is trapped in a cage of the receiving catheter. The guidewire can then be used to deliver additional implanted devices while the passing catheter, receiving catheter, or both are removed.

### INCORPORATION BY REFERENCE TO ANY PRIORITY APPLICATIONS

This application claims the benefit of priority under 35 U.S.C. § 119 (e) to U.S. Provisional Patent Application No. 63/591,376, filed Oct. 18, 2023, which is hereby incorporated by reference herein in their entireties. Any and all applications for which a foreign or domestic priority claim is identified in the Application Data Sheet as filed with the present application are hereby incorporated by reference under 37 CFR 1.57.

### BACKGROUND

### Field

Embodiments of the present application are directed to methods and catheter-based systems for connecting blood vessels or creating an access path between blood vessels.

In the body, blood vessels are naturally connected to each other to efficiently transport blood and nutrients though the body. To treat some conditions, blood vessels may be surgically or artificially connected, for example at an anastomosis. A vascular side-to-side anastomosis connects sides of each vessel together, for example to create a longer blood flow channel or to redirect blood flow. For many vascular surgeries, for example bypass grafts and organ transplants, the quality of anastomosis largely determines the success of the procedure. Typical vascular anastomosis surgery requires significant preparation to properly identify the two vessels to be joined as well as the location of the anastomosis. In some locations, for example when the blood vessels are not parallel, few locations are suitable for the anastomosis because the vessels are sufficiently close within only a small area. In these cases, for example where the vessels are generally perpendicular and/or cross at essentially a single point, a high degree of accuracy is required to correctly connect the vessels.

In other vascular surgery procedures, access to a surgical target may be gained by first accessing the bloodstream at a convenient location and navigating through vasculature to the target. For example, cardiac procedures may percutaneously access a femoral artery, femoral vein, radial artery, subclavian vein, or other vessel and approach the heart via vascular navigation. In some approaches, the distance and complexity may be reduced by leaving one vessel and entering another to create a more direct route between the percutaneous access point and the target. In some approaches, a vessel may be blocked or narrow and create navigational difficulties, which could be addressed by accessing an alternate path in a nearby vessel. Creating an access pathway between vessels may provide a beneficial alternate approach.

### SUMMARY

Disclosed below are systems and methods of connecting blood vessels or creating an access path between blood vessels. A connection or access path between blood vessels can be created via a catheter-based “kissing balloon” system. The system includes a first delivery or passing catheter and a second receiving catheter that cooperate from within two vessels to create a path between the two vessels.

In some aspects, the techniques described herein relate to a method of creating an access path, the method including: placing a passing catheter in a first blood vessel; inflating a balloon of the passing catheter within the first vessel; placing a receiving catheter in a second blood vessel; inflating a balloon of the receiving catheter within the second vessel; and deploying a piercing element from the passing catheter. Balloons on both the passing and receiving catheters can stabilize each catheter within their respective vessels. The passing catheter can be used to deliver a piercing element having a sharp distal tip for puncturing the first blood vessel. The method can include extending a hollow needle from the passing catheter to the receiving catheter. The hollow needle may be pre-shaped to bend toward the vessel wall. In other implementations, the hollow needle may be passively or actively steered toward the vessel wall. Optionally, the hollow needle can be advanced along the piercing guide to puncture the first and second vessels. The method can include extending an atraumatic guidewire within a lumen of the hollow needle to connect the first and second vessels; and capturing an end of the guidewire in a cage of the receiving catheter. The systems and methods described herein allowed for a controlled method of creating the access path. Using these methods, the vessel wall can be punctured without assistance from RF or other ablative energy, but in other implementations, ablative methods may be incorporated.

In some aspects, the method further includes puncturing the balloon of the receiving catheter with the hollow needle. In some aspects, the balloon of the receiving catheter is filled with a fluid including a contrast to provide visual confirmation of the puncture. In some aspects, the balloon of the receiving catheter is located within the cage. In some aspects, the method further includes expanding the cage.

In some aspects, the method further includes withdrawing at least the receiving catheter and leaving the guidewire connecting the first and second vessels in place. In some aspects, the method further includes withdrawing the passing catheter. In some implementations, the passing catheter or the receiving catheter can be used to deliver a treatment device like a shunt.

In some aspects, the piercing guide is pre-shaped to direct the hollow needle toward the second vessel. In some aspects, the hollow needle is pre-shaped to follow the piercing guide and extend through the first and second vessels, toward the cage. The piercing guide and/or hollow needle can include a shape memory material allowing either the piercing guide or hollow need to bend to a pre-shaped configuration when unsheathed. In some examples, the piercing guide and/or hollow needle may include one or more cut outs to facilitate bending.

In some aspects, the first vessel is a vein and the second vessel is an artery. In some aspects, the first vessel is an artery and the second vessel is a vein. But in other methods, the system may be used to between two arteries or between two veins. In some aspects, the techniques the first vessel and the second vessel are non-parallel. The two vessels do not need to be touching each other. There may be a distance between the two vessels over which the needle traverses.

In some aspects, the techniques described herein relate to a system for creating an access path between a first blood vessel and a second blood vessel. The system can include a passing catheter with a passing catheter body. The passing catheter can include one or more inflatable balloons to stabilize the passing catheter. The system can include a deployable piercing element. The deployable piercing element may include a piercing guide and/or deployable hollow needle. The system may include a guidewire, such as an atraumatic guidewire. The system may include a receiving catheter including a receiving catheter body and one or more inflatable balloons. The receiving catheter may include a deployable cage for at least partially or fully surrounding one of the inflatable balloons. The passing catheter is configured to be placed in the first vessel and the receiving catheter is configured to be placed in the second vessel. The hollow needle can be configured to be deployed along the piercing guide and to puncture the first and second vessels. The guidewire can be configured to be extended within a lumen of the hollow needle. The cage can be configured to capture a distal end of the guidewire.

In some aspects, the cage is outside at least one of the one or more inflatable balloons of the receiving catheter. In some aspects, the hollow needle is further configured to puncture at least one of the one or more balloons of the receiving catheter. When punctured, the balloon may release contrast to verify puncture. In some aspects, the piercing guide exits at a location spaced apart from a distal tip of the passing catheter, for example a sidewall of the passing catheter body at an exit point.

In some aspects, the one or more balloons of the passing catheter includes a stabilizing balloon proximal of the exit point. In some aspects, the one or more balloons of the passing catheter further includes a second stabilizing balloon distal to the exit point.

In some aspects, the piercing guide is pre-shaped to direct the hollow needle toward the second vessel. In some aspects, the hollow needle is pre-shaped and configured to follow the piercing guide and extend through the first and second vessels toward the cage when deployed. In some aspects, the passing catheter body includes a multi-lumen tube.

In some aspects, the one or more balloons of the receiving catheter includes a contrast balloon inside the cage and at least one stabilizing balloon along the receiving catheter body.

In some aspects, the system further includes an implant configured to be placed over the guidewire and between the first vessel and the second vessel to connect the first and second vessels. In some aspects, the implant is a shunt.

For purposes of summarizing the disclosure, certain aspects, advantages and novel features of several implementations have been described herein. It is to be understood that not necessarily all such aspects, advantages, and novel features are achieved or required in accordance with any particular implementation of the technology disclosed herein. Thus, the implementations disclosed herein can be implemented or carried out in a manner that achieves or optimizes one advantage or group of advantages as taught herein without necessarily achieving other advantages that can be taught or suggested herein.

### BRIEF DESCRIPTION OF THE DRAWINGS

Certain features of this disclosure are described below with reference to the drawings. The illustrated implementations are intended to illustrate, but not to limit, the implementations. Various features of the different disclosed implementations can be combined to form further implementations, which are part of this disclosure.

FIGS. 1-4 illustrate a method of connecting blood vessels.

FIG. 5 illustrates an implementation of a system for connecting blood vessels.

FIGS. 6A-6B illustrate implementations of a piercing element.

FIG. 7A-7B illustrate implementations of a guidewire with an atraumatic distal tip.

FIG. 8 illustrates an implementation of a distal end of a passing catheter.

FIG. 9 illustrates an implementation of an inflatable balloon inside a cage of a receiving catheter.

FIG. 10 illustrates an implementation of a connection system.

FIG. 11 illustrates an implementation of a passing catheter.

FIG. 12 illustrates an implementation of a piercing element and guidewire.

FIG. 13 illustrates an implementation of a pre-shaped piercing guide of a piercing element.

FIG. 14 illustrates another implementation of a pre-shaped hollow needle.

FIGS. 15-17 illustrate an implementation of a method of connecting blood vessels.

FIG. 18 illustrates an implementation of the system for connecting blood vessels.

FIG. 19 illustrates visualization of inflated balloons of an implementation of the system.

FIGS. 20A-B illustrate an implementation of a receiving cage.

FIG. 21 illustrates an implementation of a balloon with a flat edge.

FIG. 22 illustrates an implementation of a cylindrical balloon.

FIG. 23 illustrates an implementation of a spherical balloon.

FIGS. 24-25 illustrate implementations of multi-lumen catheter bodies.

### DETAILED DESCRIPTION

The systems and methods described herein are for connecting blood vessels or creating an access path between blood vessels, for example to deploy a guidewire 30. The system may include a passing catheter 10 and a receiving catheter 50. The passing catheter 10 may include one or more balloons 40. The system may include a piercing element 20 extending through the passing catheter 10. The piercing element 20 may include one or more structure for piercing the vessel wall. The piercing element 20 may be an elongate structure with a sharp distal tip capable of piercing the vessel wall without the assistance of ablative energy. For example, the piercing element 20 may include a needle as described herein. The needle may be a hollow needle. The hollow needle may be pre-shaped to bend toward the vessel wall when released from the passing catheter 10. The guidewire 30 may extend through the hollow needle. In some implementations, the piercing element 20 may include piercing guide for guiding the needle toward the vessel wall as described further below. The piercing guide may be a stylet of shape memory material shaped to guide the needle toward the vessel wall. For example, the piercing guide may be pre-shaped to bend toward the vessel wall when released from the passing catheter 10. In other implementations, the piercing guide and/or the hollow needle may be actively steered toward the vessel wall.

The receiving catheter 50 may include one or more balloons 60. The receiving catheter 50 may include a cage 80. The cage 80 may provide one or more functions, for example trapping or snaring the piercing element 20 and/or guidewire 30 or protecting a balloon 60. The cage 80 may include a shape memory material.

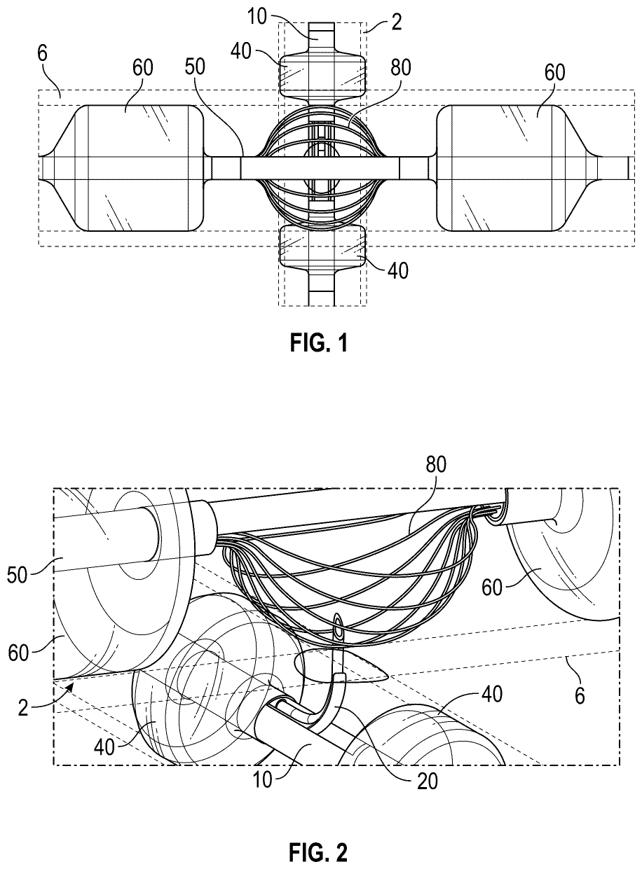

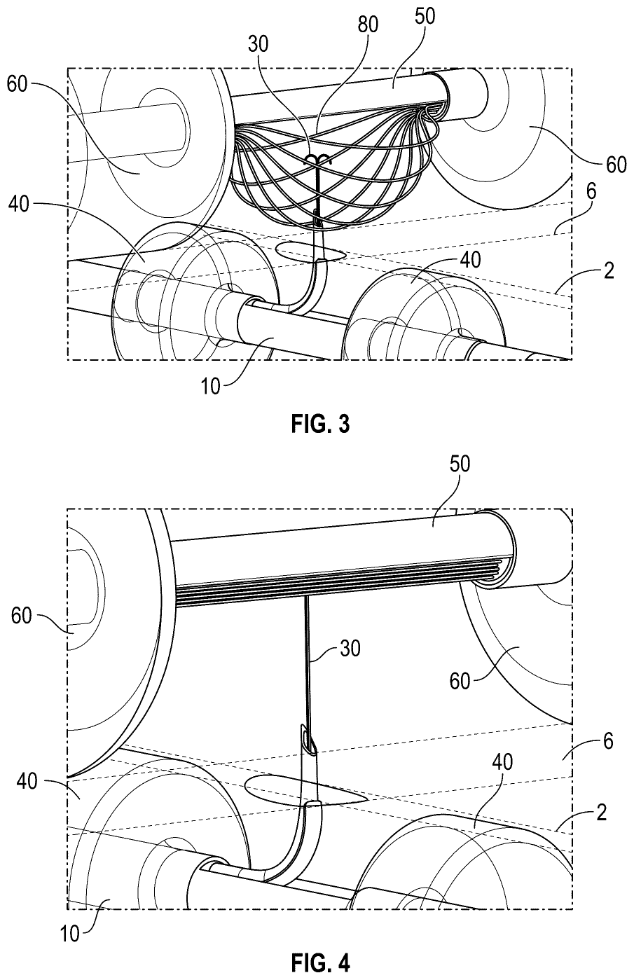

FIGS. 1-4 illustrate a method of connecting blood vessels. The method may include placing a passing catheter 10 in a first vessel 2, inflating at least one balloon 40 of the passing catheter 10 within the first vessel 2, and placing the receiving catheter 50 in a second vessel 6, as shown in FIG. 1. The first vessel 2 and second vessel 6 may be in contact with each other or may be spaced apart by a distance. As illustrated in FIG. 2, a piercing element 20, which may include a piercing guide, may be deployed from the passing catheter 10. The deployment may include extending a hollow needle component of the piercing element 20 from the passing catheter 10 toward the receiving catheter 50 along the piercing guide to puncture the first vessel 2 and the second vessel 6. As shown in FIG. 3, a guidewire 30 may be extended within the lumen of the hollow needle to connect the vessels 2, 6, and a distal end of the guidewire 30 may be captured in a cage 80 of the receiving catheter 50. As shown in FIG. 4, collapsing the cage 80 may trap the guidewire 30, allowing withdrawal of both the passing catheter 10 and the receiving catheter 50 to leave the guidewire 30 connecting the vessels in place.

Systems for connecting blood vessels are described below. Features of the systems resemble the system discussed above in many respects or may include similar features. Accordingly, numerals used to identify similar features of the system are incremented by a factor of one hundred (100). For example, the piercing element 120 may include any of the features of piercing element 20 and vice versa. This numbering convention generally applies to the remainder of the figures. Any component or step disclosed in any embodiment in this specification can be used in other embodiments.

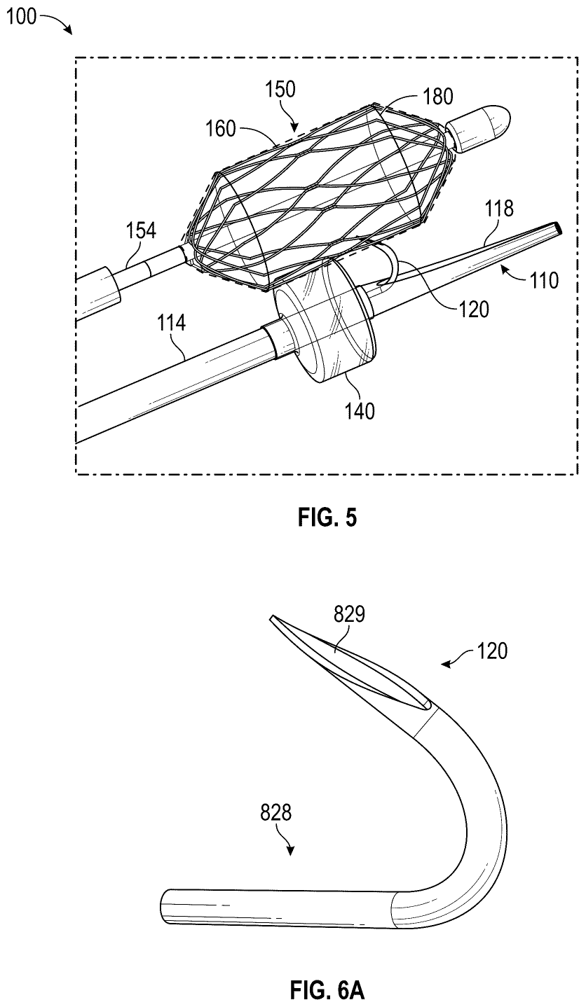

A system for connecting blood vessels, for example system 100 shown in FIG. 5, may be used to perform the method shown in FIGS. 1-4. For example, the system 100 may include a passing catheter 110 and receiving catheter 150. The passing catheter 110 can include a passing catheter body 114. The passing catheter body 114 may include an integral or separately connected distal end 118. The passing catheter 110 may include one or more balloons, for example passing catheter balloon 140 carried by passing catheter body 114. The passing catheter balloon 140 may be inflated in a first vessel to be joined at or near a passing location or point where the vessels are to be joined. The balloon 140 may be inflated against the vessel wall of the passing vessel to center and stabilize the passing catheter 110 within the passing vessel. In some implementations, the balloon 140 may be inflated with a fluid or fluid solution, for example a liquid, gas, or appropriate mixture. In some implementations, a balloon 140 may be inflated with saline, contrast, or a contrast solution. As illustrated, the passing catheter 110 includes a single balloon 140 spaced apart from a distal tip of the passing catheter 110, for example proximal of the distal end 118. The single balloon 140 may be proximal of an opening where the piercing element 120 exits the passing catheter body 114. In some implementations, the passing catheter 110 includes two balloons, as described below. For example, the passing catheter 110 may include two balloons 140 configured to be arranged with one balloon on each side of the passing location.

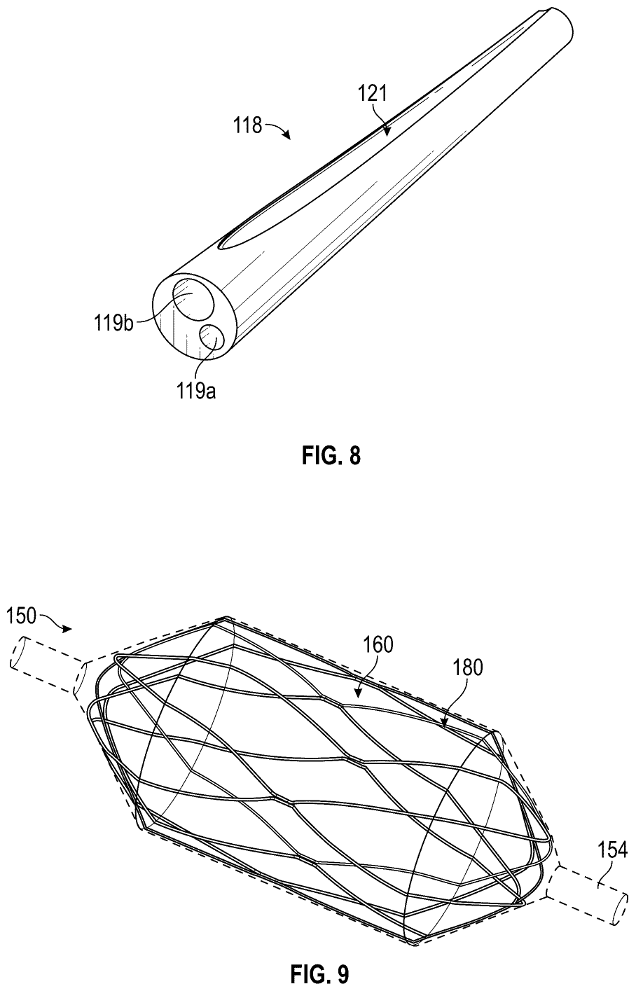

The passing catheter 110 also includes a deployable piercing element 120, for example piercing element 120 shown in FIG. 5. The piercing element 120 may exit the passing catheter 110 at the passing location. As illustrated, the piercing element 120 may exit the passing catheter body 114 proximal of the distal tip of the passing catheter body 114. For example, the passing catheter 110 may include a distal end 118 (see FIG. 8) with a passageway that allows the piercing element 120 to bend toward the vessel at a location spaced apart from the distal tip of the catheter 110. In other configurations, the piercing element 120 may extend from a sidewall or distal tip of the passing catheter body 114.

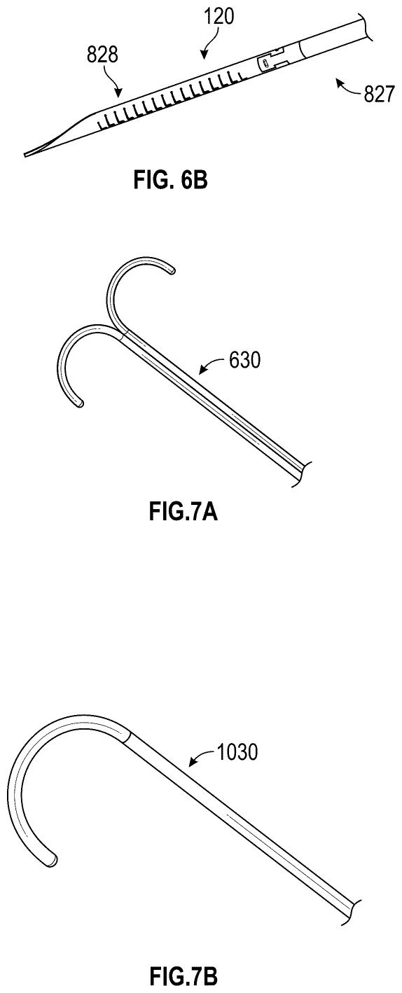

In some implementations, the piercing element 120 can include hollow needle 828 as shown in FIG. 6A. The distal end of the hollow needle 828 allows the lumen 829 to exit at a sharp distal point or bevel. As shown in FIG. 6A, the hollow needle 828 may be at least partially pre-shaped to a configuration when exposed from the passing catheter body 114. A pre-shaped hollow needle 828 may provide additional rigidity and puncture force. In some implementations, for example hollow needle 828, the hollow needle may be pre-shaped to bend at an angle of greater than or equal to 90 degrees such that the tip is at an angle of less than 90 degrees relative to the shaft as shown in FIG. 6A. In some implementations, the hollow needle is pre-shaped to include an angle of approximately 90 degrees (see FIG. 14). As shown in FIG. 6B, a distal portion of the piercing element 120 may include one or more cutouts to allow the needle 828 to bend relative to the proximal shaft 827 to a configuration as shown in FIG. 6A.

After the first and second vessel walls are pierced by the piercing element 120, a guidewire 30 can be passed through the lumen 829 of the hollow piercing needle 828. The needle 828 is then retracted, leaving the guidewire in place connecting the first and second vessels.

As discussed above and shown in FIGS. 2-3, guidewire 30 is deployed through the lumen of a hollow needle, and the distal end of the guidewire 30 is trapped by a cage 80. Guidewire 30 may include an atraumatic distal tip with one or more distal hooks, barbs, or other capture features to assist in trapping the distal end of the guidewire 30 in the cage 80. In some implementations, for example guidewire 630 shown in FIG. 7A, the guidewire may include an atraumatic distal tip with two proximally-facing hooks. In some implementations, guidewire 30 may include 3, 4, 5, or more hooks. In some implementations, for example as shown in FIG. 7B, a guidewire 1030 may include a single hook, barb, or other capturing feature facing proximally from an atraumatic distal tip.

In some implementations, for example as shown in FIG. 5, the piercing element 120 exits the passing catheter body 114 at a location proximal of the distal tip. In some implementations, a distal end 118 of the passing catheter 110 may be configured to facilitate smooth entry and exit of a piercing element 120 and/or guidewire 30, 630, 1030. For example, as illustrated in FIG. 8, a distal end 118 may include an elongate opening 121 configured to assist deployment and retraction of piercing element 120, for example to allow piercing element 120 to deploy and assume a pre-set shape. As illustrated, the distal end 118 can include multiple lumens. The distal end 118 can include a first lumen 119a for delivering the passing catheter 110 over a guidewire or to provide access for other instruments. In some implementations, the first lumen 119a may be utilized to inflate the balloon 140. The distal end 118 can include a second lumen 119b for delivery of the piercing element 120. The second lumen can have a distal opening that is recessed from the distal tip of the distal end 118. The second lumen can open to the elongate opening 121. The elongate opening 121 can be recessed passageway extending at least partially along a length of the distal end 118.

As illustrated in FIGS. 1-4, the receiving catheter 50 can include one or more balloons 60 inflated in a second vessel 6 to be joined. The one or more balloons 60 may be positioned within or outside of the cage 80. In some implementations balloons 60 can be inflated at or near the passing location within the second vessel 6. In some implementations, the one or more balloons 60 may be configured to be inflated against the vessel wall to center and stabilize the receiving catheter in the receiving vessel. As illustrated in FIGS. 1-4, the receiving catheter may include two balloons 60. A first balloon 60 is positioned distal of the cage 80 and/or a second balloon 60 is positioned proximal of the cage 80. In some implementations, at least one balloon 60 may be inflated with fluid. In some implementations, a balloon 60 is filled with fluid contrast and/or saline to assist in visualization. When inflatable balloon 60 is punctured, the contrast is released into the second vessel 6, which may be visually confirmed. The receiving catheter 50 can include a cage 80 for capturing a guidewire 30 from the passing catheter 10, as shown in FIG. 3.

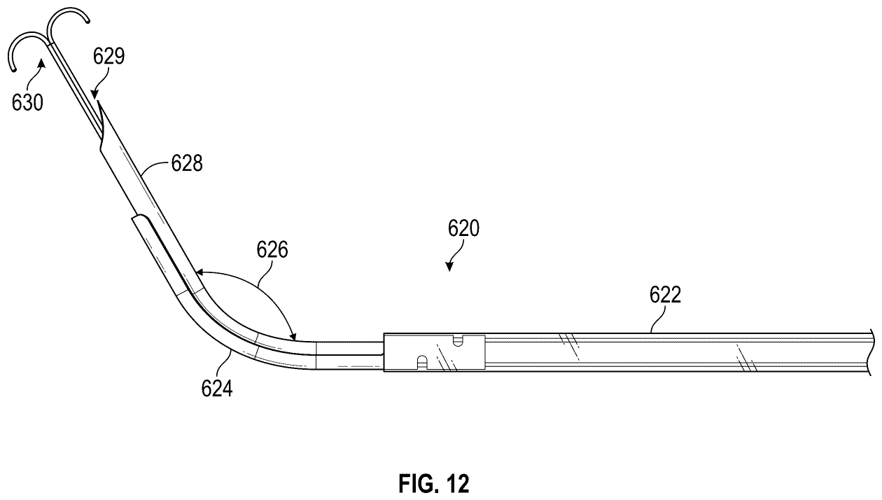

In some implementations, the receiving catheter 50 may include at least one balloon 60 inside the cage 80. For example, passing catheter 150 partially illustrated in FIG. 9 may include a catheter body 154 carrying an inflatable balloon 160 inside a cage 180. In some implementations, cage 180 at least partially or fully surrounds a circumference of inflatable balloon 160. In some implementations, inflation of inflatable balloon 160 expands cage 180 within the second vessel 6. In some implementations, the shape memory cage 180 is deployed, for example by withdrawing a constraining sheath, and the balloon 160 is inflated within the expanded cage 180. In some implementations, cage 180 is separately expandable within the second vessel 6. For example, cage 180 may be deployed or expanded via pull wire, push rod, power screw, or other suitable mechanism. In some implementations, cage 180 is pre-shaped to expand to a deployed configuration after being released from catheter 150. In some implementations, inflatable balloon 160 and cage 180 are each generally symmetric. In some implementations, cage 180 surrounds inflatable balloon 160. As illustrated in FIG. 9, the cage 180 and/or the balloon 160 may have a generally cylindrical central portion with tapered ends. In some implementations, for example as shown in FIG. 12, inflatable balloon 160 surrounds the catheter body 154, and cage 180 surrounds inflatable balloon 160. In some implementations, inflatable balloon 160 may be inflated to occlude blood flow within the second vessel 6.

In some implementations of use, the piercing element 120, pierces the wall of the second vessel 6 and enters the cage 180 before piercing inflatable balloon 160. Piercing inflatable balloon 160 may be visually confirmed via imaging. For example, in some implementations, inflatable balloon 160 is inflated with fluid (appropriate gas or liquid) to occlude blood flow within the second vessel 6. When the inflatable balloon 160 is punctured, the inflatable balloon 160 will deflate and allow blood to resume flow within the second vessel 6, which may be visually confirmed. In some implementations, inflatable balloon 160 is filled with contrast, or a mix of contrast and fluid, for example saline. When inflatable balloon 160 is punctured, the contrast is released into the second vessel 6, which may be visually confirmed. In some implementations, the cage 180 is outside the inflatable balloon 160, meaning puncturing the inflatable balloon 160 also confirms that the distal tip of piercing element 120 is within the cage 180.

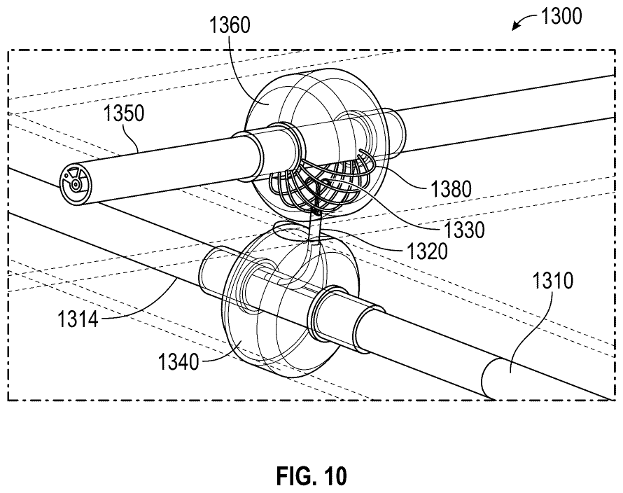

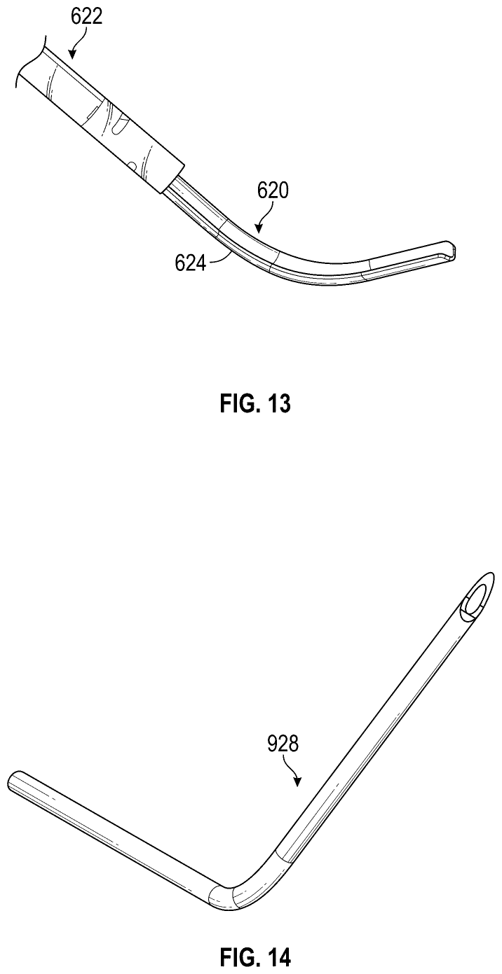

FIG. 10 illustrates an implementation of a system 1300 that can be deployed similar to the method described above with respect to FIGS. 1-4 and may include any of the features of the system 100. The system 1300 can include a passing catheter 1310 including one balloon 1340 and a receiving catheter 1350 including one balloon 1360. Passing catheter 1310 may be similar to passing catheters 10, 110 in some respects. For example, passing catheter 1310 may include a catheter body 1314 with one or more inflatable balloons 1340. The catheter body 1314 may be a multi-lumen catheter body. The passing catheter 1310 may include a piercing element 1320. The piercing element 1320 may include a pre-shaped piercing guide and/or hollow needle. The system 1300 may include an atraumatic guide wire 1330 to be advanced through the catheter body 1314. In some implementations, inflatable balloon 1340 may be inflated in a first vessel to center and stabilize passing catheter 1310 within the vessel and to occlude blood flow. In some implementations, for example as shown in FIG. 13, inflatable balloon 1340 is located along passing catheter 1310 where piercing element 1320 exits the sidewall of catheter body 1314. The passing catheter 1310 may carry a single inflatable balloon 1314. The single inflatable balloon 1314 may be positioned proximal of a distal tip of the catheter body 1314. The inflatable balloon 1314 may be positioned over a location the piercing element 1320 exits the catheter body 1314. In some implementations, inflatable balloon 1340 may include a radial channel to allow piercing element 1320 to exit the inflatable balloon 1340 without puncturing the balloon. In some implementations, piercing element 1320 may be configured to puncture the inflatable balloon 1340, for example with a hollow needle, before puncturing the wall of the first vessel.

Receiving catheter 1350 may be similar to receiving catheters 50, 150 in some respects. For example, receiving catheter 1350 may include a catheter body, inflatable balloon 1360, and/or cage 1380 as described herein. In some implementations, for example system 1300 illustrated in FIG. 10, receiving catheter 1350 may include a single balloon 1360. In some implementations, inflatable balloon 1360 may be inflated in a second vessel to center and stabilize receiving catheter 1350 within the second vessel and to occlude blood flow. In some implementations, balloon 1360 may be inflated with contrast as described herein. In some implementations, receiving catheter 1350 may include cage 1380. As illustrated in FIG. 10, cage 1380 may only partially surround the balloon 160. The cage 180 may be a semi-circular cage that deploys in an arc around the receiving catheter 1350. In some implementations, the cage 1380 deploys in an arc of about 180 degrees. In some implementations, the cage 1380 deploys in an arc of less than 360 degrees and may be oriented toward the passing catheter 1310.

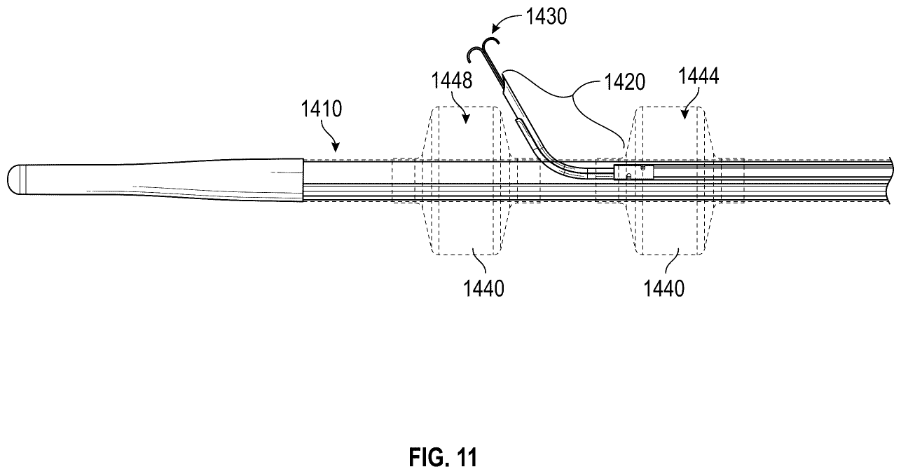

In some implementations, a passing catheter 10 may include two balloons 40. For example, as illustrated in FIG. 11, a passing catheter 1410 may include two balloons 1440 including a proximal balloon 1444 and a distal balloon 1448. Piercing element 1420 may be arranged between proximal balloon 1444 and distal balloon 1448 to deploy guidewire 1430. Balloons 1444, 1448 may be similar to balloons 40, 140, 1340 in some or all respects. For example, with reference to FIGS. 1-4, balloons 1444, 1448 may be inflated within the first vessel 2 to stabilize and center passing catheter 1410 and may be inflated to occlude blood flow through the first vessel 2. In some implementations, each of proximal balloon 1444 and distal balloon 1448 may separately occlude blood flow. In some implementations, they may cooperate together to occlude or slow blood flow in vessel 2. In some implementations, distal balloon 1448 may be configured to assist deployment of piercing element 1420, for example by bracing piercing element 1420 as it punctures the first vessel, second vessel, and/or receiving balloon.

In some implementations, the piercing element 1420 includes a piercing guide for guiding a sharp tip of the piercing element 1420 toward the vessel wall. The piercing guide may be pre-shaped and/or steered toward the vessel wall. For example, the piercing guide may be passively or actively steered toward the vessel wall. In other implementations, the piercing guide may include shape memory material and pre-shaped to bend toward the vessel wall. For example, FIG. 12 illustrates a pre-shaped piercing guide 624 of piercing element 620. The piercing guide 624 may be constrained in the passing catheter 1410 and assumes a bent shape after exiting the side of the passing catheter 1410. As shown in FIG. 12, the pre-shaped piercing guide 624 may be attached to a shaft 622 or other controllable mechanism to allow deployment and/or rotation of pre-shaped piercing guide 624. In some implementations, pre-shaped piercing guide 624 includes an essentially tubular section attached to a shaft 622 with a semi-tubular section at a distal end of pre-shaped piercing guide 624, as shown in FIGS. 11 and 12.

In some implementations, pre-shaped piercing guide 624 is pre-shaped to assume an angle 626. In some implementations, angle 626 is at least about 15 degrees and/or less than or equal to about 160 degrees, for example, from about 15-30 degrees, 20-45 degrees, 40-90 degrees, 80-95 degrees, 90-120 degrees, 110-130 degrees, 120-160 degrees, and other angles inclusively within these ranges. In some implementations, the angle 626 is approximately 90 degrees.

A hollow piercing needle of the piercing element 1420, for example hollow piercing needle 628 or hollow piercing needle 928 illustrated in FIG. 14, can then be deployed from the passing catheter 1410 into the piercing guide 624, as shown in FIG. 2. The piercing needle 628 is configured to pierce both a wall of the first vessel 2 and a wall of the second vessel 6 as the hollow piercing needle 628 is extended from the passing catheter 1410. The piercing needle 628 also punctures a portion of the receiving catheter, as described in any of the embodiments above. In some implementations, hollow piercing needle 628 is a pre-shaped needle that assumes an angled or curved shape after deployment from the shaft 622 or passing catheter 1410. In some implementations, a hollow piercing needle 628 may assume an angle, for example the same angle 626, after deployment. In some implementations, hollow piercing needle 628 is pre-shaped to match a pre-shaped piercing guide such as guide 624. Hollow piercing needle 628 can include a lumen 629 to deploy an atraumatic guidewire 630.

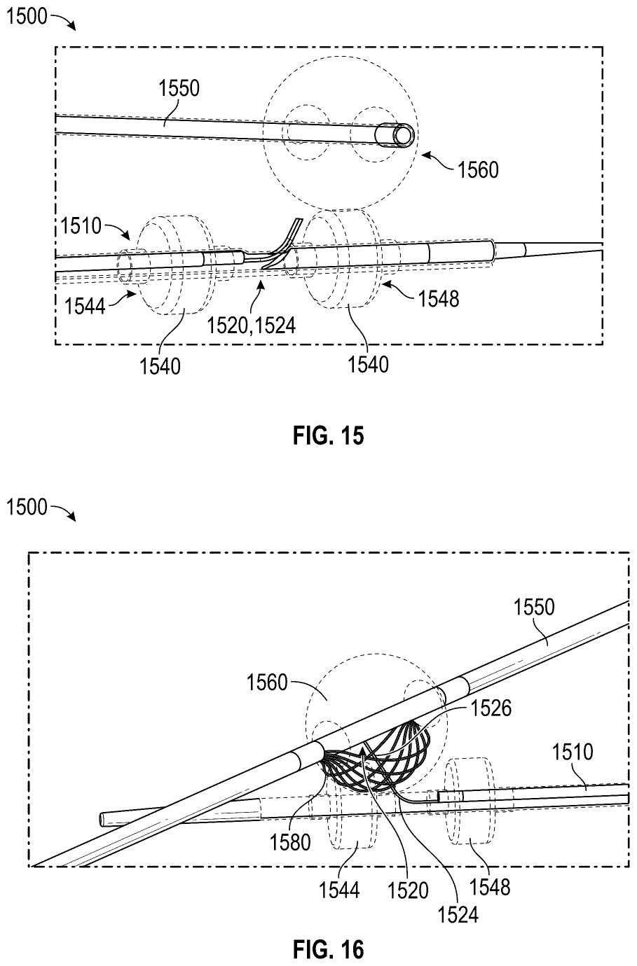

FIGS. 15-17 illustrate an implementation of a method of using a system 1500 for connecting blood vessels. System 1500 may include a passing catheter 1510 and a receiving catheter 1550. Passing catheter 1510 may be similar to passing catheters 10, 110, 1310, 1410 in some or all respects, and may include, for example two balloons 1540 such as proximal balloon 1544 and distal balloon 1548, and piercing element 1520 between the balloons. A receiving catheter 1550 may include an inflatable balloon 1560 and deployable cage 1580. As shown in FIG. 15, passing catheter 1510 can be inserted in a first vessel (not shown for clarity). The balloon 1560 may be shaped differently than the passing catheter balloons 1540. For example, the passing catheter balloon 1540 may be more cylindrical for stabilization; whereas, the receiving catheter balloon 1560 may be more spherical. The two balloons 1540 can be inflated to occlude blood flow and/or stabilize passing catheter 1510 within the first vessel. Receiving catheter 1550 can be inserted in a second vessel (not shown for clarity). Inflatable balloon 1560 can be inflated with a contrast solution to stabilize receiving catheter 1550 within the second vessel and may also occlude blood flow. One of the balloons 1540, for example distal balloon 1548 can be aligned with inflatable balloon 1548, but may be positioned elsewhere relative to the passing catheter balloons. Piercing element 1520 can be deployed from a sidewall of passing catheter 1510. In some implementations, a piercing guide, such as pre-shaped piercing guide 1524, may be deployed first, as illustrated in FIG. 15.

As shown in FIG. 16, a cage, for example partial arc shape memory cage 1580, may be deployed from the receiving catheter 1550. As discussed elsewhere, deployable cage 1580 may be inside inflatable balloon 1560, outside inflatable balloon 1560, or within the wall of inflatable balloon 1560. As further illustrated in FIG. 16, a needle, for example pre-shaped hollow needle 1526 can be deployed from passing catheter 1510 along pre-shaped piercing guide 1524. Hollow needle 1526 can be deployed to puncture the first vessel, the second vessel, and the inflatable balloon 1560, also entering the cage 1580, as shown. The shaped piercing guide 1524 is extended to oppose the wall of the first vessel towards the second vessel. The preloaded piercing needle 1526 is extended from the piercing guide 1524 to pierce both the first vessel wall and the second vessel wall. As the needle 1526 is further extended, it also enters the cage 1580 and punctures the balloon 1560 filled with contrast in the second vessel. Therefore, piercing of both vessel walls can be confirmed via fluoroscopy or other visualization, since the pierced balloon will release dye into the second vessel and will be visible fluoroscopically as a “puff” of contrast and/or visible deflation of the contrast balloon 1560.

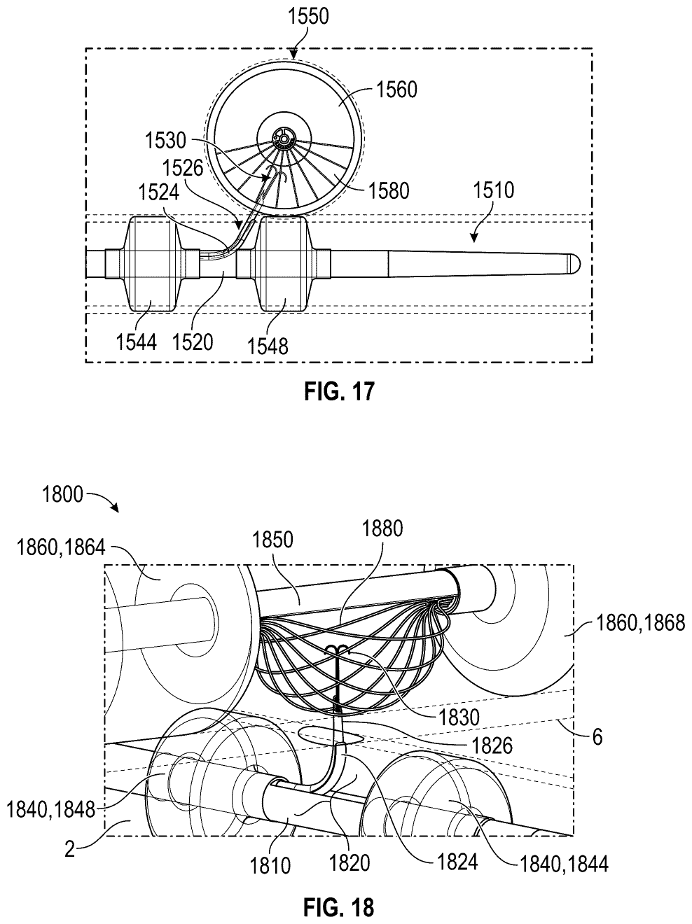

FIG. 17 illustrates deployment of a guidewire, for example atraumatic guidewire 1530 with two distal hooks. As shown, guidewire 1530 can be deployed within the lumen of pre-shaped hollow needle 1526 to pass out of the first vessel and into the second vessel. As the guidewire 1530 exits the distal piercing end of pre-shaped hollow needle 1526, it also enters the deployable cage 1580. In some implementations, the distal hooks of guidewire 1530 become entangled in the struts of the cage 1580. In some implementations, the distal end of guidewire 1530 becomes entangled in the deployable cage 1580 when the cage is collapsed. In some implementations, deployable cage 1580 is collapsed by a pull wire, by advancing a constraining sheath from receiving catheter 1550, or other appropriate mechanism. In some implementations, the cage 1580 entangles the end of the guidewire 1530 by manipulating the guidewire and/or cage, for example by twisting, hooking, retracting, anchoring, etc. In some implementations, the guidewire 1530 and cage 1580 may be magnetically attracted. As discussed above, trapping the distal end of guidewire 1530 in the deployable cage 1580, and thereby in receiving catheter 1550, allows the guidewire to remain in place connecting the first and second vessels. After the guidewire 1530 is passed through the lumen of the hollow piercing needle 1526, the needle 1526 can be retracted. The cage 1580 captures the distal end of the guidewire 1530 to hold it in place while one or both catheters 1510, 1550 are withdrawn.

Once the guidewire 1530 is captured in the receiving catheter 1550, the receiving catheter 1550 can be removed from the receiving vessel, which may also externalize the guidewire 1530 on the receiving side, and the passing catheter 1510 can be removed from the first vessel. This leaves the guidewire 1530 in place between the vessels, creating a delivery path for additional devices. In some implementations, the passing catheter 1510 is used to deliver the additional devices over the guidewire 1530 before the passing catheter 1510 is removed from the first vessel. In some implementations, the cage 1580 is removed from the lumen of the receiving catheter 1550, which remains in place over the guidewire 1530, so the receiving catheter 1550 may be used to deliver additional devices over the guidewire 1530 before the receiving catheter 1550 is removed from the second vessel.

FIG. 18 illustrates an implementation of the system, for example system 1800, wherein the passing catheter 1810 includes a proximal balloon 1844 and a distal balloon 1848. Piercing element 1820 includes piercing guide 1824 and hollow needle 1826 containing atraumatic guidewire 1830. Passing catheter 1810 may be similar to passing catheters 10, 1410, 1510 in some or all respects. Receiving catheter 1850 includes a proximal balloon 1864 and a distal balloon 1868 with a cage 1880 between the balloons.

As noted above, the system is used to provide a connection between a first vessel and a second vessel. In use, the passing catheter is percutaneously inserted into a first vessel to be joined and the receiving catheter is percutaneously inserted into the second vessel to be joined. The stabilizing balloons of both the first catheter and second catheter are inflated to stabilize the catheters and directly oppose or “kiss” each other at or near the passing location, for example as shown in FIG. 19 with passing balloon 1940 and receiving balloon 1960. As illustrated, the balloons, for example, balloons 40, 60, 140, 160, 1340, 1440, 1444, 1448, 1540, 1544, 1548, 1560, 1844, 1848, 1864, 1868, 1940, 1960 may all be optionally inflated with contrast or contrast solution to assist visualization.

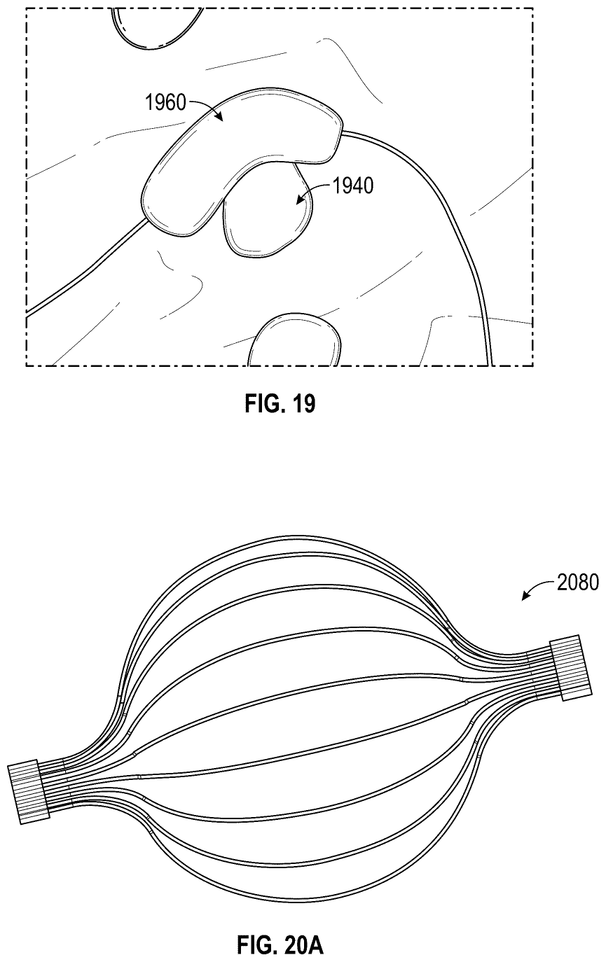

As mentioned above, in some implementations the cage 80 may be a tubular, cylindrical, round, oval, or spherical cage. For example, cage 180 may symmetrically surround catheter body 154. In some implementations, cage 80 may be hemispherical or dome shaped. For example, cage 2080 shown in FIGS. 20A-B may be deployed in an arc of approximately 180 degrees around the catheter body (for example as illustrated in FIGS. 10 and 18). In some implementations, a cage 80 may be deployed in an arc of less than 180 degrees, for example as illustrated in FIGS. 2, 3, 16, and 17. As noted above, the cage 80 may be oriented toward the passing catheter 10 when deployed. In some implementations, a balloon 60 may be the same shape as the cage 80.

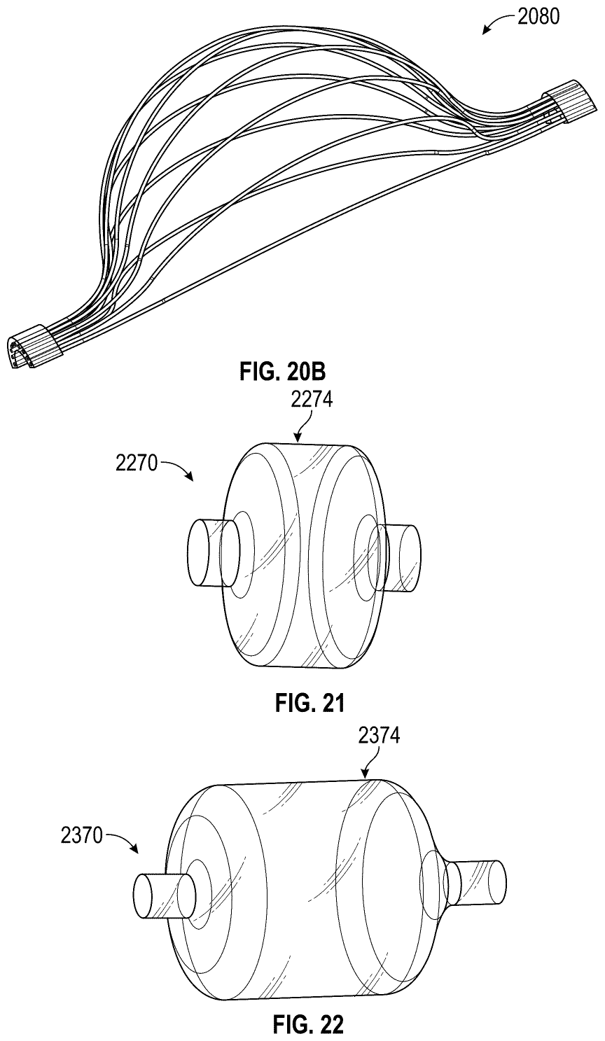

In some implementations, the balloons of both catheters, for example balloons 40, 60, 140, 160, 1340, 1440, 1444, 1448, 1540, 1544, 1548, 1560, 1844, 1848, 1864, 1868, 1940, 1960, may include features to assist in mating and/or visualization. In some implementations, the balloons may also have pre-shaped flat or mating sides to better approximate each other. For example, as illustrated in FIG. 21, balloon 2270 may include a flat edge 2274 to provide a large flat surface area for mating. As illustrated in FIG. 22, balloon 2370 may include a flat edge 2374 that is longer than a diameter of the balloon 2370, thus providing a more cylindrical shape. In some implementations, for example balloon 2470 illustrated in FIG. 23, the balloon may be essentially spherical to allow for mating against any surface. In some implementations, the balloons may include radio-opaque marks, such as rings, arrows, lines, stripes, or other appropriate markings to allow visualization and alignment. In some implementations, the balloons may include magnetic portions to attract the balloons toward each other. Aligning the balloons also brings the two vessels as close together as possible to minimize the distance between vessels and can optimize passing of additional components.

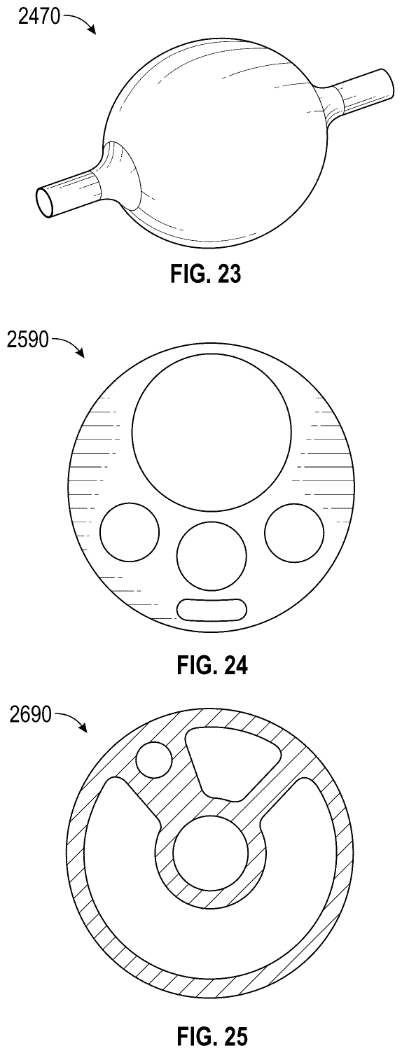

In some implementations, catheters described herein may include multi-lumen tubular bodies. For example, as shown in FIG. 24, a catheter may include a multi-lumen tubular body 2590, which may include several lumens for steering, suction, irrigation, inflation, deflation, sensors, and passing of the various components described above. In some implementations, each component may be provided in a different lumen. In some implementations, some or all components (for example, piercing guide, hollow needle, and guidewire) may be provided within a single lumen. As illustrated in FIG. 25, a catheter may include a multi-lumen tubular body 2690, which may include several lumens including an arcuate lumen for delivery and retraction of the cage 80.

Referring again to FIGS. 1-4, In some implementations, the first vessel 2 is an artery and the second vessel 6 is a vein, allowing the passing catheter 10 to extend the piercing element 20 from a high-pressure vessel into a lower-pressure vessel. In some implementations, the first vessel 2 is a vein and the second vessel 6 is an artery. This arrangement allows the passing catheter 10 to extend the piercing element 20 out of a vein with low pressure into an artery with a higher pressure, which may be easier to puncture without collapsing or sliding to the side of the needle of the piercing element 20. In some implementations, the first and second vessels are essentially parallel. For example, the system can be used to provide an arteriovenous (AV) fistula for a dialysis patient. In some implementations, the first and second vessels are not parallel or otherwise aligned, and intersect at approximately a single point. For example, the system can be used to provide an access path between a pulmonary artery and an azygos vein and/or deliver a treatment device or implant such as a shunt as described in more detail in U.S. Provisional Application No. 63/591,382, filed Oct. 18, 2023, as well as U.S. Patent Application No. ______ [Attorney Docket No. INQB.030A] and titled “SYSTEMS AND METHODS FOR TREATING HEART FAILURE BY REDIRECTING BLOOD FLOW IN THE AZYGOS VEIN,” filed on the same day as the present application, both of which are hereby incorporated by reference in their entireties.

### Additional Considerations and Terminology

Features, materials, characteristics, or groups described in conjunction with a particular aspect, implementation, or example are to be understood to be applicable to any other aspect, implementation or example described herein unless incompatible therewith. All of the features disclosed in this specification (including any accompanying claims, abstract and drawings), or all of the steps of any method or process so disclosed, may be combined in any combination, except combinations where at least some of such features or steps are mutually exclusive. The protection is not restricted to the details of any foregoing implementations. The protection extends to any novel one, or any novel combination, of the features disclosed in this specification (including any accompanying claims, abstract and drawings), or to any novel one, or any novel combination, of the steps of any method or process so disclosed.

While certain implementations have been described, these implementations have been presented by way of example only and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some implementations, the actual steps taken in the processes illustrated or disclosed may differ from those shown in the figures. Depending on the implementation, certain of the steps described above may be removed, others may be added. For example, the actual steps or order of steps taken in the disclosed processes may differ from those shown in the figure. Depending on the implementation, certain of the steps described above may be removed, others may be added. Furthermore, the features and attributes of the specific implementations disclosed above may be combined in different ways to form additional implementations, all of which fall within the scope of the present disclosure.

Although the present disclosure includes certain implementations, examples and applications, it will be understood by those skilled in the art that the present disclosure extends beyond the specifically disclosed implementations to other alternative implementations or uses and obvious modifications and equivalents thereof, including implementations which do not provide all of the features and advantages set forth herein. Accordingly, the scope of the present disclosure is not intended to be limited by the described implementations, and may be defined by claims as presented herein or as presented in the future.

Conditional language, such as “can,” “could,” “might,” or “may,” unless specifically stated otherwise, or otherwise understood within the context as used, is generally intended to convey that certain implementations include, while other implementations do not include, certain features, elements, or steps. Thus, such conditional language is not generally intended to imply that features, elements, or steps are in any way required for one or more implementations or that one or more implementations necessarily include logic for deciding, with or without user input or prompting, whether these features, elements, or steps are included or are to be performed in any particular implementation. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Likewise, the term “and/or” in reference to a list of two or more items, covers all of the following interpretations of the word: any one of the items in the list, all of the items in the list, and any combination of the items in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain implementations require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain implementations, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

What is claimed is:

1. A method of creating an access path, the method comprising:
placing a passing catheter in a first blood vessel;
inflating a balloon of the passing catheter within the first vessel;
placing a receiving catheter in a second blood vessel;
inflating a balloon of the receiving catheter within the second vessel;
deploying a piercing guide from the passing catheter;
extending a hollow needle from the passing catheter to the receiving catheter along the piercing guide to puncture the first and second vessels;
extending a guidewire within a lumen of the hollow needle to connect the first and second vessels; and
capturing an end of the guidewire in a cage of the receiving catheter.

2. The method of claim 1, further comprising puncturing the balloon of the receiving catheter with the hollow needle.

3. The method of claim 1, wherein the balloon of the receiving catheter is filled with a fluid comprising a contrast.

4. The method of claim 2, wherein the balloon of the receiving catheter is located within the cage.

5. The method of claim 1, further comprising expanding the cage.

6. The method of claim 1, further comprising withdrawing at least the receiving catheter and leaving the guidewire connecting the first and second vessels in place.

7. The method of claim 6, further comprising withdrawing the passing catheter.

8. The method of claim 1, wherein the piercing guide is pre-shaped to direct the hollow needle toward the second vessel.

9. The method of claim 8, wherein the hollow needle is pre-shaped to follow the piercing guide and extend through the first and second vessels, toward the cage.

10. The method of claim 1, wherein the first vessel is a vein and the second vessel is an artery.

11. The method of claim 1, wherein the first vessel is an artery and the second vessel is a vein.

12. The method of claim 1, wherein the first vessel and the second vessel are non-parallel.

13. A system for creating an access path between a first blood vessel and a second blood vessel, the system comprising:
a passing catheter comprising a passing catheter body, one or more inflatable balloons, a deployable piercing guide, and deployable hollow needle;
a guidewire; and
a receiving catheter comprising a receiving catheter body, one or more inflatable balloons and a deployable cage,
wherein:
the passing catheter is configured to be placed in the first vessel and the receiving catheter is configured to be placed in the second vessel;
the hollow needle is configured to be deployed along the piercing guide and to puncture the first and second vessels;
the guidewire is configured to be extended within a lumen of the hollow needle; and
the cage is configured to capture a distal end of the guidewire.

14. The system of claim 13, wherein the cage is outside at least one of the one or more inflatable balloons of the receiving catheter.

15. The system of claim 13, wherein the hollow needle is further configured to puncture at least one of the one or more balloons of the receiving catheter.

16. The system of claim 13, wherein the piercing guide exits a sidewall of the passing catheter body at an exit point.

17. The system of claim 16, further wherein the one or more balloons of the passing catheter comprises a stabilizing balloon proximal of the exit point.

18. The system of claim 17, wherein the one or more balloons of the passing catheter further comprises a second stabilizing balloon distal to the exit point.

19. The system of claim 13, wherein the piercing guide is pre-shaped to direct the hollow needle toward the second vessel.

20. The system of claim 19, wherein the hollow needle is pre-shaped and configured to follow the piercing guide and extend through the first and second vessels toward the cage when deployed.

21. The system of claim 13, wherein the passing catheter body comprises a multi-lumen tube.

22. The system of claim 13, wherein the one or more balloons of the receiving catheter comprises a contrast balloon inside the cage and at least one stabilizing balloon along the receiving catheter body.

23. The system of claim 13, further comprising an implant configured to be placed over the guidewire and between the first vessel and the second vessel to connect the first and second vessels.

24. The system of claim 23, wherein the implant is a shunt.
